(19) United States
(12) Patent Application Publication
(10) Pub. No.: US 2015/0175970 A1
(43) Pub. Date: Jun. 25, 2015

(54) CELLS FOR THERAPY OF THE HEART, METHOD OF OBTAINING A CELL PREPARATION, AND CELL PREPARATION

(71) Applicant: Charite Universitatsmedizin Berlin, Berlin (DE)

(72) Inventors: Marion Haag, Berlin (DE); Jochen Ringe, Hohen Neuendorf (DE); Michael Sittinger, Berlin (DE); Carsten Tschope, Berlin (DE)

(73) Assignee: Charite Universitatsmedizin Berlin, Berlin (DE)

(21) Appl. No.: 14/642,816

(22) Filed: Mar. 10, 2015

Related U.S. Application Data
(62) Division of application No. 12/544,760, filed on Aug. 20, 2009.
Application No. 12/544,760 is a continuation of application No. PCT/EP2008/052027, filed on Aug. 20, 2008.

(30) Foreign Application Priority Data
Aug. 20, 2007 (DE) 10 2007 008 650.6

(51) Int. Cl. C12N 5/077; G01N 33/569

(52) U.S. Cl. CPC C12N 5/0657; G01N 33/56966; C12N 2509/00; C12N 2501/06; C12N 2506/1307

(57) ABSTRACT

Fibroblast-like cells obtained from heart muscle biopsies, which are CD90 negative, CD105 positive, CD117 negative and/or CD166 positive as well as cell preparations of such cells for therapy of heart diseases, as well as a method for providing the latter. The cells are characterized by a good cultivability in cell culture. Furthermore a method for obtaining the cells and cell preparations according to the invention are disclosed.

### CROSS-REFERENCE TO RELATED APPLICATION

This is a divisional of U.S. patent application Ser. No. 12/544,760, filed Aug. 20, 2009, which was a continuation, under 35 U.S.C. §120, of international patent application PCT/EP2008/052027, filed Feb. 20, 2008; the application further claims the priority, under 35 U.S.C. §119, of German patent application No. □10 2007 008 650.6 filed Feb. 20, 2007; the prior applications are herewith incorporated by reference in their entirety.

### BACKGROUND OF THE INVENTION

The invention relates to cells and a cell preparation for therapy of heart diseases as well as methods for producing the cells and cell preparations according to the invention.

Diseases of the heart and cardiovascular system belong to the most important causes of disease in industrialized societies. Among them, cardiac insufficiency, as a cause or consequence of a pathology caused by other factors, is one of the most common diseases. The number of cases in Europe alone lies within the double-digit millions.

Among experimental therapies, which are discussed with respect to cardiac insufficiency, is also cell therapy. The basis of this therapeutic approach is the expectation that the weakened myocardium should be strengthened by the immigration and the proliferation of the therapeutically applied cells into the myocardium and that its functional efficiency should be increased.

The production of adult stem cells from the heart constitutes a topic of scientific research. Whilst the fundamental ability for regeneration of cardiac tissue has been questioned for a long time, several approaches for regeneration of weakened myocardium either from stem cells or from not fully differentiated stem cell-like cells are presently under examination and in part under clinical development.

Pittenger et al. have characterized mesenchymal stem cells (Science 284, 143-147); which show inter alia a CD90 positive phenotype.

Wang et al. (International Journal of Cardiology 109 (2006) 74-81) showed the differentiation of mesenchymal stem cells of the rat to differentiated heart cells in co-culture with fully differentiated heart cells. The cells obtained thereby are also CD90 positive. Similar results are found by Moscoso et al. (Transplantation Proceedings, 37, 481-482 (2005)) in porcine cells.

Messina et al. (Circulation Research, Oct. 29, 2004, pp. 911-924; WO2005/012510) describe a method for isolation and cultivation of heart cells from biopsies. The cells isolated there are inter alia c-kit/CD117 positive.

A fundamental problem in the application of cell therapy for the therapy of heart diseases is to obtain sufficient amounts of cells for therapeutic application.

### SUMMARY OF THE INVENTION

The problem underlying the present invention is to obtain, in a simple method from cell material that is relatively easily accessible outside the body, a cell preparation which is suitable to be applied to a patient suffering from cardiomyopathy and other heart diseases, for instance infarcts and their aftereffects, with the aim of improving patients' cardiac output.

According to the invention, this problem is solved by an isolated mammalian cell having the features: the cell is a cell that was proliferated in cell culture from a primary culture of a tissue sample obtained from a mammal; the cell is a fibroblast-like cell; the cell is CD90 negative; the cell is CD105 positive; and the cell is CD117 negative.

Further, this problem is solved by a method for obtaining a cell preparation, wherein

a) a tissue sample obtained from mammalian heart muscle tissue is subjected to a time limited digestion by one or more connective tissue digesting enzymes,
b) the time limited digested tissue sample is cultivated in a first cell culture step under conditions suitable for the culture of mammalian cells in cell culture medium in a cell culture container having a solid surface,
c) cells grown out of the tissue sample adhering to the solid surface are detached in a passage step by means of limited proteolysis, isolated and are cultured again, diluted in a cell culture medium,
d) step c) is repeated at least twice, and
e) the cell culture medium of either the first cell culture step, the passage step or of both steps does not contain cardiotrophin, thrombin or mercaptoethanol.

Further, this problem is solved by a cell preparation comprising the cells according to the invention and by a cell preparation which can be generated by a method according to the invention.

### DETAILED DESCRIPTION OF BEST MODE AND SPECIFIC/PREFERRED EMBODIMENTS OF THE INVENTION

According to one embodiment of the present invention a cell or a preparation of a plurality of cells can be provided for therapy of heart disease by bringing biopsy material obtained from a patient or a tissue sample from cells of a patient into a primary culture and proliferation through several passages. Obtaining cells from a primary culture, which was established from a heart muscle biopsy, for instance from a pinhead-sized or even smaller biopsy sample, is preferred. The biopsy materials are “plucked” by means of a small “caliper”. The culture conditions are described in example 1.

According to an embodiment of the present invention the method is distinguished from the one described by Messina at al. (see above) by the absence of cardiotrophin and thrombin in the cell culture medium.

Furthermore, no 2-mercaptoethanol is used in the medium. According to one embodiment of the present invention, the cells grown out of biopsy material are trypsinized. Thus, all steps described by Messina et al. for “harvesting” cells are omitted. The cells are not proliferated in coated cell culture dishes, since these cells adhere very well. Further cultivating of the cells is thus also distinguished, since the “cardiospheres” described in Messina et al. do not adhere, i.e., they do not grow on solid surfaces in culture, such as for instance the bottom of a cell culture vessel, a wall of a cell culture bottle, a film in a nutrient medium or a porous matrix for cell culture. Preferred cells of the present invention do adhere.

According to one embodiment of the present invention, a method is provided, which allows obtaining a cell population that is characterized by a series of specific characteristics: the cells are fibroblast-like, i.e. elongated, spindle-shaped cells having a morphology as shown in FIG. 1b) and in FIG. 3, and they grow adherently under cell culture conditions shown in example 1.

Surprisingly, these cells are negative with respect to staining by the marker CD90 characteristic for mesenchymal stem cells and fibroblasts.

In this context it is apparent that no homogeneous cell population can be obtained by creating, for example, a primary culture from a heart biopsy material. It is also apparent that for therapeutic application, the application of a plurality of cells, i.e., a cell preparation, would currently be considered. Such cell preparation can be obtained by a cell culture method as described in example 1. The cells contained therein will differ with respect to their phenotype, since they are not grown from a homogeneous population as described above. Accordingly, the characteristic features of a cell preparation according to the invention are not be stated as absolutely “positive” or “negative” concerning a cell marker, but with respect to the whole cell population.

Therefore, according to one embodiment of the present invention, the cell preparation can be characterized in that a plurality of the cells contained therein is CD90 negative. This means that at least 50% of the cells contained in this population are not stained any more by a standard dye marker in the FACS, for instance the dye marker stated in the examples, as cells typically known to the person skilled in the art as CD90 negative cells. CD90 (Thy-1) is a marker for thymus cells, hematopoietic stem cells, NK cells and endothelial cells, fibroblasts and myofibroblasts. Preferably, according to an embodiment of the present invention, the cell preparation is at least 80%, more preferably 90% CD90 negative. Even more preferred are cell preparations which are more than 95%, 98% or 99% CD90 negative.

In order to achieve a preferred homogeneity with respect to CD90 negativity of the cell preparation, the method for obtaining the cell preparation can comprise according to one embodiment of the present invention a purification step, in which the cells are selected with respect to their expression of CD90. For this purpose, methods of fluorescence-based cell sorting (FACS sorting) in suitable devices are known for example. Thereby, cells marked by a fluorescence-marked antibody against the respective antigen are automatically separated in a capillary into a negative and a positive population.

Furthermore, separation by means of magnetic separation is known. Thereby, cells are separated in a very strong magnetic field by means of retention of the antigen positive cells by antibody-coupled magnetic particles.

Means and methods for separation of cells regarding their expression of antigens are known to experts. According to an embodiment of the present invention the cell preparation may also be obtained using a method which, as an alternative or in addition to the separation with respect to CD90 negativity, comprises further separation steps, which select regarding further possible characteristics of the cell preparation:

According to an embodiment of the present invention the cell preparation can be characterized in that a plurality of the cells contained therein is CD105 positive. This means that at least 50% of the cells contained in this population are stained in the FACS by means of a standard dye marker, for instance the dye marker stated in the examples. CD105 is a typical marker for endothelial cells and mesenchymal cells. Preferably, according to an embodiment of the present invention, the cell preparation is at least 90%, more preferably at least 98% CD105 positive.

Furthermore, according to an embodiment of the present invention, the cell preparation is characterized in that a majority of the cells contained therein is CD117 negative. This means that at least 50% of the cells contained in this population are not stained anymore by a standard dye marker in the FACS, for example the dye marker stated in the examples, as cells typically known to the person skilled in the art as CD117 negative cells (negative test). CD117 (c-kit) is a stem cell marker. Preferably, according to an embodiment of the present invention, the cell preparation is 60%, more preferably at least 70% CD117 negative.

Also, according to an embodiment of the present invention, the cell preparation can be characterized in that the majority of cells contained therein are CD166 positive. This means that at least 50% of the cells contained in this population are stained by a standard dye marker, for example the dye marker stated in the examples, in the FACS. CD166 is the acronym for “activated leukocyte cell adhesion molecule (ALCAM)”, a marker typical for mesenchymal stem cells from the bone marrow. Preferably, according to an embodiment of the present invention, the cell preparation is at least 60%, more preferably at least 70% CD166 positive.

Furthermore, according to an embodiment of the present invention, the cell preparation can be characterized in that a majority of the cells contained therein is CD34 negative and CD45 negative. This means that at least 50% of the cells contained in this population are not stained anymore in the FACS by a standard dye marker, for example the dye marker stated in the examples, as cells typically known to the person skilled in the art as CD34-negative or CD45-negative, respectively (negative test). CD34 and CD45 are both stem cell markers. Preferably, the cell preparation is at least 60%, more preferably at least 70% CD34 and CD45 negative.

Furthermore, according to an embodiment of the present invention, the cell preparation can be characterized in that the majority of the cells contained therein is desmin positive after myogenic induction (see examples), and/or positive with respect to antibodies against cardiac smooth muscle myosin. Preferably, the cell preparation is at least 60%, more preferably at least 70% desmin positive and/or myosin positive.

Besides the stated characterization as CD90 negative, the cell preparations may comprise according to an embodiment of the present invention all stated characteristics concerning the fraction of CD105 positive, CD117 negative, CD166 positive, CD34/35 negative cells.

In this respect, the statement of “positivity” or “negativity” can be related to two different populations. This shall be explained by means of a cell preparation denoted as “60% CD105 positive, 90% CD90 negative”:

On one hand, this denotation can relate to a cell preparation in which 60% of all cells are CD105 positive. In the same way the characterization 90% CD90 negative would relate to the whole population. Since here the definition of positivity or negativity does not relate to the whole population, respectively, this criterion shall be denoted as “global characterization.”

On the other hand, a cell preparation in which 60% of the CD90 negative cells are CD105 positive, can be described in this way. This characterization, in case of which a second criterion is only applied to cells which fulfill the first criterion, shall be denoted here as “cumulative characterization”.

The amount of cells contained in a cell preparation, which is definitely CD90 negative and CD105 positive, may possibly differ depending on whether the global or the cumulative characterization is used as a definition. This difference will be larger the more criteria are considered, especially in the case of criteria near 50%.

According to an embodiment of the present invention, a cell preparation shall be defined by means of the stated criteria with respect to expression of CD90, CD105, CD117, CD166 and CD34/45 in a global as well as cumulative characterization, namely by means of stated percentages, wherein the cumulative characterization is preferred.

The method allows for the first time to obtain cell preparations having a cell count of more than 10¹⁰, actually up to 10¹³⁻¹⁴ cells for therapeutic application to the heart.

According to the invention, cells can be administered to the patient as a pharmaceutical preparation. At first, it is preferred in this connection, to administer autologous cell preparations to human patients. Known immunological problems contravene the application of heterologous cell preparations; there are however realistic indications, in case of which even heterologous cell preparations are to be preferred to, for instance, the transplantation of a donor heart. Particularly, the cell preparation offers the great advantage that, for example, cell surface proteins, which allow the identification of the heterologous cells by the patients' immune system, can be concertedly blocked during the preparation method, so that the heterologous cell preparation does not comprise or comprises significantly reduced the known immunological disadvantages of a heterologous transplant.

For the application of the cells according to the invention a plurality of known methods exists. Gyongyosi et al. describe inter alia (J. Kardiol 2004, 11 (Supp B; pp 22-24) the direct injection of cells into the heart muscle by means of an intra-myocardial catheter-based injection. Since fibroblasts comprise tropism, a systemic application is further conceivable.

### DEFINITIONS

Cells in the sense of this invention are somatic cells of mammals, □

particularly humans. According to the invention, cells and cell preparations in accordance with the independent claims can be used for preparation of a pharmaceuticals for disease therapy. In this respect, the preparation of an autologous cell or cell preparation as well as of an allogeneic cell or cell preparation is possible. Autologous cells or cell preparations are obtained by taking biopsy material from the same patient to whom they are returned. Allogeneic cells or cell preparations are obtained from a different person.

Cells or cell preparations can also be obtained from biopsy material taken from a donor heart. These cells may then be extracorporeally proliferated and stored, in order to be given to the transplanted patient in case of a medical necessity.

Denoting a cell population as “fibroblast-like” in the sense of the present invention means that the majority of the cells display a substantially spindle-shaped appearance under the microscope. Spindle-shaped means that the majority of the cells, according to the invention, comprise an elongated shape, for instance that the cells at confluence have a length of 150-250 μm. At lower confluence however, one also finds cells having a length of merely 60 μm up to over 350 μm (elongated shape). The width of the cells can lie within the range of 13-20 μm, wherein 9 μm and more than 30 μm are also possible.

The cells are also distinguished by their characteristic shape as shown in FIG. 1b) (A-E) and FIG. 3. Furthermore, fibroblast-like cells in the sense of the present invention are characterized in that they stick or adhere in culture to the bottom of the cell culture container according to the cell culture conditions stated in the examples and can be detached from the bottom of the cell culture container by means of trypsinization. The more confluent (dense) the cell culture, the more uniformly fibroblast-like the cell shape.

The denotation of a cell as “negative” in relation to a tissue marker, such as for example a protein of the CD (“cluster of differentiation”)-series, e.g. CD90, CD105, CD117, CD166, means that the cell is not stained by means of a prescribed staining with a marked antibody against the denoting marker in a way, that the cell yields concerning the order of magnitude a comparably strong signal with respect to the labeling of the respective antibody in the FACS (fluorescence-based cell sorting device) or fluorescent microscopy as a cell that is regarded by the experts as “positive” in relation to the respective surface marker with the same antibody and under comparable staining conditions.

The same holds true in an analogous fashion for staining methods inside the cell.

Known, unambiguous examples for cell types denoted as positive or negative concerning two substantial markers in relation to the present invention are mesenchymal stem cells. These are positive for CD 105 and CD 90 and negative for CD 34 and 45.

According to an embodiment of the present invention a primary tissue sample obtained from a living organism or an organism dead for less than 24 h is brought into culture in a nutrient medium in a cell culture container in a suitable manner as known to a person skilled in the art, for example at 37° Celsius, 95% humidity and 5% CO2. This culturing step includes a partial digestion of the tissue sample by means of proteases. Preferred in this connection are trypsin-EDTA and collagenase IV. Alternatively, the digestion may be conducted without trypsin-EDTA.

This culture is denoted as “primary culture”. During growth cells are observed at regular intervals and harvested/isolated after reaching a predefined cell density. According to an embodiment of the present invention, cells grow out of the biopsy material (outgrowth culture) and are then harvested or isolated. These cells are in turn cultured and passaged each time the cells cover 70-90% of the bottom of the cell culture container (confluence of 70-90%).

Transferring cells from one culture container to another, wherein most of the times a dilution of the cells occurs, is denoted as a passage. This term is a synonym for sub-culture and should not be confused with the passage in virology (Toni Lindl, “Cell- and tissue culture” 4^{th} edition, Spektrum Verlag, p. 255). Typically, the cells are thereby detached from the bottom of the cell culture container by applying trypsin (“trypsinization”) and are sown again at a density of 5000-6000 cells/cm². Thus, the cells are transferred from one passage into the next passage.

The highest density arrangement of adherent cells possible as a mono layer in culture is denoted as “confluence” (see also Lindl, ibid., p. 253).

Diseases which can be treated with the cells, cell preparations and pharmaceuticals of the independent claims or indications for the application of the inventive cells and cell preparations may be inter alia heart diseases such as the ischemic cardiomyopathy with good and bad ejection fraction, inflammatory cardiomyopathy with good and bad ejection fraction, diastolic dysfunction, aortic valve defects (stenosis, insufficiency), mitral valve defects (stenosis, insufficiency), right heart insufficiency, bradycardiac and tachycardiac dysrhythmia including AV blocks and atrial fibrillation, coating of coronary stents, diabetic cardiopathy, collagenosis having cardiac involvement, familiar cardiomyopathies, virally induced myocarditis and cor hypertensivum.

According to an embodiment of the present invention, an isolated mammalian cell is provided which is characterized by the following features:

the cell is a cell proliferated in cell culture from a primary culture of a tissue sample obtained from a mammal,
the cell is a fibroblast-like cell, and the cell is CD90 negative.

Preferably, the cell was proliferated by means of at least three passages in cell culture. The cell can be of human origin.

According to a preferred embodiment, the isolated mammalian cell is CD105 positive. According to a preferred embodiment the isolated mammalian cell is CD105 positive, CD117 negative, CD166 positive, CD34/45 negative.

Furthermore, a method for obtaining a cell preparation is provided, in which:

a) a tissue sample obtained from a mammal is subjected to a time limited digestion by one or more connective tissue digesting enzymes,
b) the time limited digested tissue sample is cultivated in a first cell culture step under conditions suitable for culture of mammalian cells in a cell culture medium, in a cell culture container having a solid surface,
c) cells grown out of the tissue sample adhering to the solid surface are detached in a passage step by means of limited proteolysis, isolated and are cultured again, diluted in a cell culture medium,
d) step c) is repeated at least twice.

According to a preferred embodiment, the tissue sample obtained in the method contains heart muscle tissue. More preferably, the connective tissue digesting enzyme activity is trypsin-EDTA or collagenase IV or a combination of both activities and the duration of the time limited digestion is less than 10 minutes at an activity of 0.05 to 0.25 u/500 ml for trypsin and/or 0.2 to 4.5 units/ml [u/ml] for collagenase IV.

According to a preferred embodiment of the method, the first cell culture step has a duration of 7 to 15 days. It is further preferred, that the passage step is conducted at a confluence of the cells adhering to the solid surface of 70% or larger, and/or that the cell culture medium of the first cell culture step or the passage step or of both steps does not contain cardiotrophin, thrombin or mercaptoethanol.

According to a preferred embodiment of the method, the cell preparation obtained in the previously described steps a) to d) is subjected to a step of myogenic induction, as a consequence of which cells positive for staining with α-desmin and/or myosin antibodies can be obtained.

According to a preferred embodiment of the method, the cell preparation obtained in the steps a to d is subjected to a purification step, in which:

the cells contained in the cell preparation are brought into contact with molecules capable of binding specific cell surface markers and
those cells, to which the molecules capable of binding specific cell surface markers have bound, are separated.

Thereby, the molecules capable of binding specific cell surface markers can be antibodies against CD90, CD105, CD117, CD166, CD34 and/or CD45.

In the purification step, molecules capable of binding specific cell surface markers can be bound to magnetic particles and retained during the purification step in a magnetic field. The cells may also be separated by means of fluorescence-activated cell sorting (FACS).

Furthermore, a cell preparation is provided that contains cells that are CD105 positive, CD117 negative, CD166 positive, CD34/45 negative, α-desmin positive and/or myosin positive.

Furthermore, a cell preparation obtained by means of the afore-characterized purification step is provided. Preferably, the cells contained therein are more than 90% CD90 negative, more than 90% CD105 positive and more than 50% CD117 negative. More preferably, the cells contained therein are more than 95% CD90 negative, more than 95% CD105 positive, more than 60% CD117 negative and more than 50% CD166 positive. Furthermore, the cells in the cell preparation characterized in this way may be more than 90% CD34 negative and more than 90% CD45 negative.

Furthermore, a cell preparation is provided, in which the cells contained therein are more than 95% CD90 negative, and more than 90% of the CD90 negative fraction of the cell preparation consists of CD105 positive cells. More preferably, the cells contained therein are more than 95% CD90 negative, and more than 90% of the CD90 negative fraction of the cell preparation is CD105 positive and more than 50% of the CD90 negative fraction is CD117 negative. More preferably, the cells contained therein are more than 98% CD90 negative, and the CD90 negative fraction of the cell preparation is more than 95% CD105 positive, more than 60% CD117 negative and more than 50% CD166 positive.

Furthermore, a cell preparation is preferred in case of which the cells contained therein are more than 95% CD90 negative, and the CD90 negative fraction of the cell population is more than 90% CD105 positive and the fraction that is both CD90 negative and CD105 positive is more than 60% CD117 negative.

The afore-characterized cells or cell preparations can be used for producing a pharmaceutical for therapy of heart diseases, particularly cardiomyopathy.

### BRIEF DESCRIPTION OF THE DRAWINGS

FIG. 1A shows biopsy material in a cell culture container and a centimeter scale laid underneath the container.

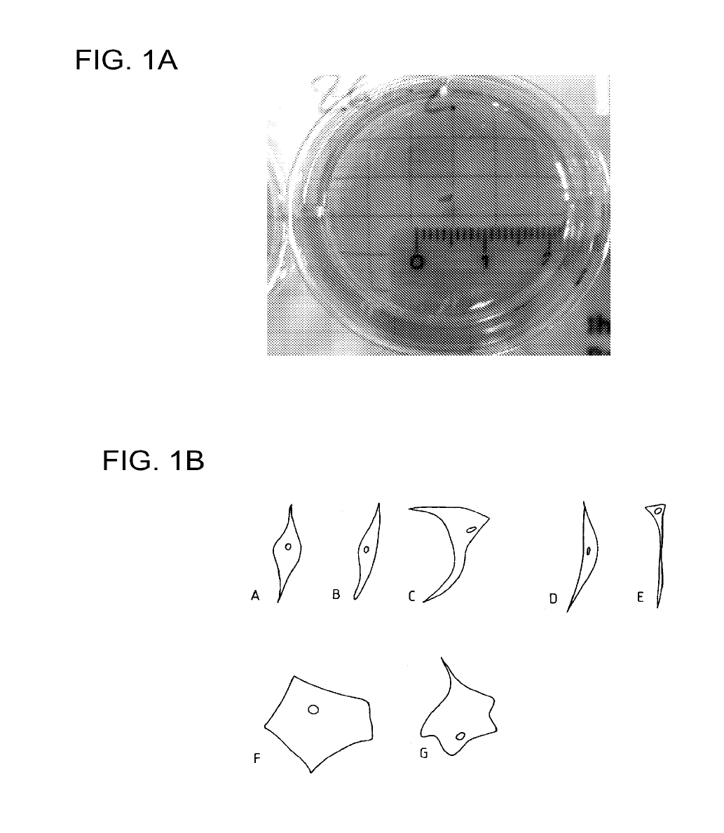

FIG. 1B shows in A-E schematic cell shapes, which may comprise the fibroblast-like cells according to the invention. F and G show cell shapes, which differ from the fibroblast-like cells according to the invention.

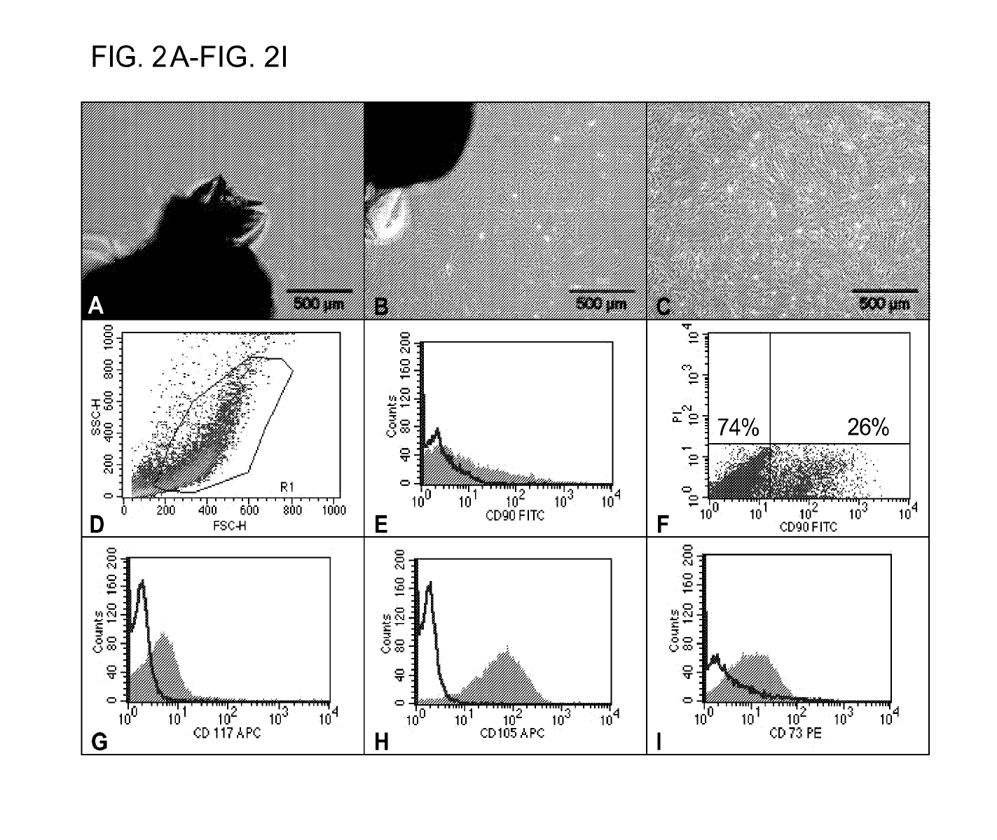

FIG. 2A-FIG. 2I shows morphology and FACS analysis of the cells or cell preparations according to the invention. FIG. 2A and FIG. 2B: Adherent cells grown in culture (FIG. 2A: after 5 days in culture), (FIG. 2B: after 15 days in culture). FIG. 2C: cell culture of the harvested cells in passage 4 after 5 days in culture. FIG. 2D-FIG. 2I: Histograms of FACS analysis, namely: FIG. 2D forward versus sideward stray light, FIG. 2E and FIG. 2F FACS analysis for CD90, CD117 (FIG. 2G), CD105 (FIG. 2H) and CD73 (FIG. 2I).

FIG. 3 shows photographic pictures of the fibroblast-like cells according to the invention under a microscope, namely cells of the lot 21-1E4 P1 day 6 (top); 16-1 E2 P3 (center); 17-2 E2 P1 day 6 (lower left-hand side) and 16-1 E3 P1 confluent (lower right-hand side).

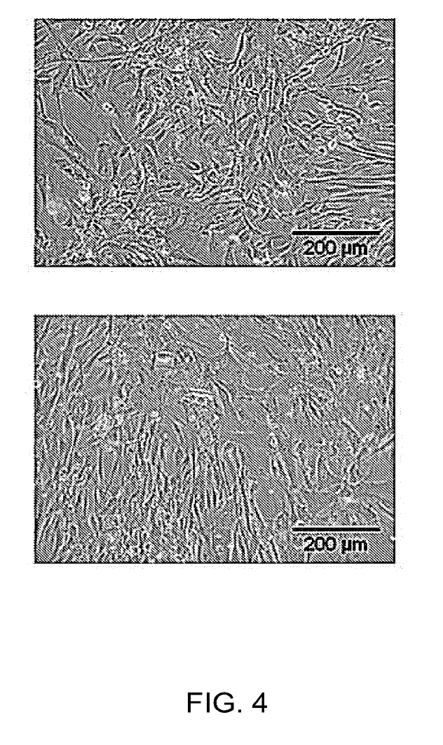

FIG. 4 shows photographic pictures of comparative examples which do not comprise the shape of the fibroblast-like cells according to the invention under the microscope, namely cells of the lot 16-4E2 primary day 6 (top) and 16-1 E3 P3 (center).

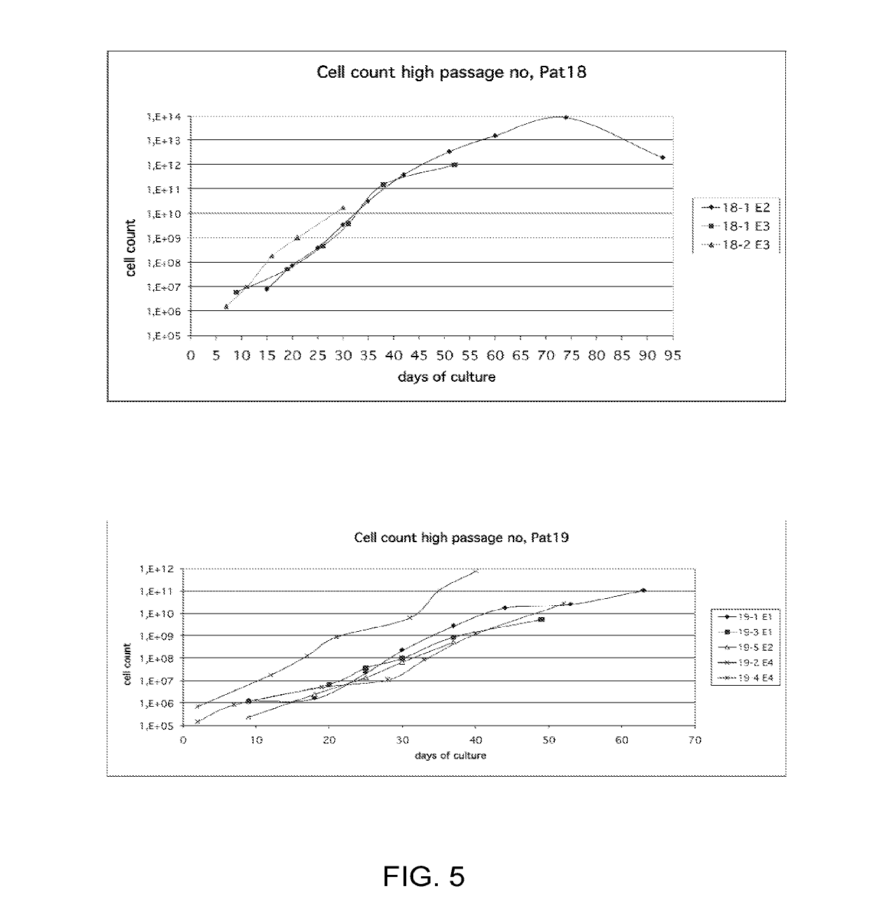

FIGS. 5 and 6 show examples of the growth kinetics of example cultures.

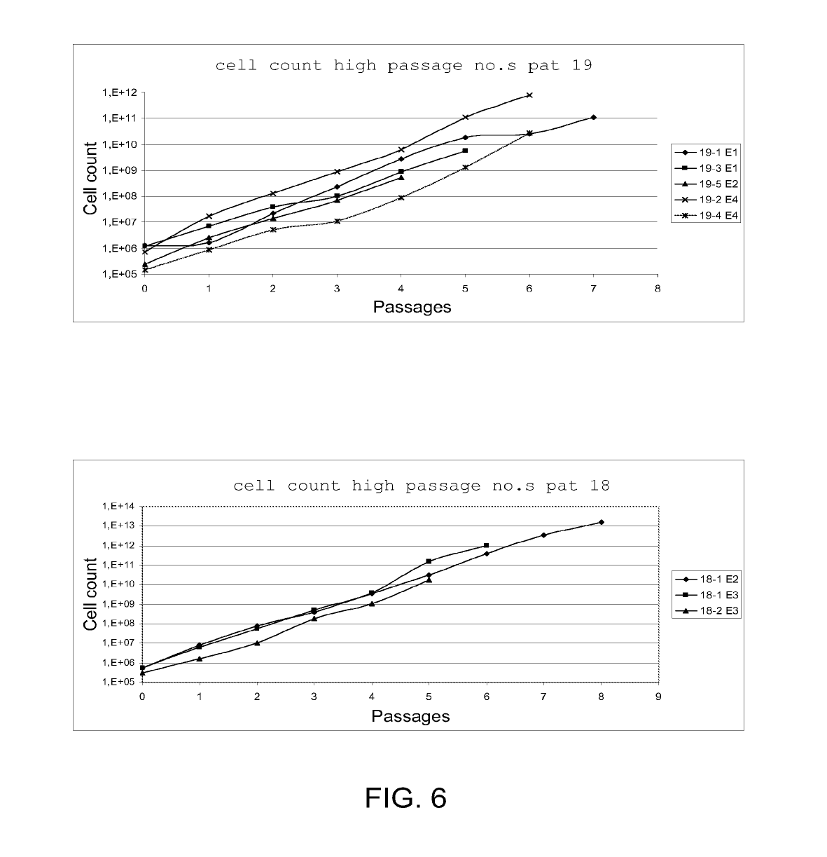

FIGS. 7A (left column) and 7B (right column) shows fluorescent microscopy pictures of cells positive for the antibodies against α-sarcomeric actin (upper row; A2172, monoclonal anti actin (α-sarcomeric) antibody) (Sigma Aldrich) secondary antibody: goat a mouse Cy3 (Dianova115-165-003)) as well as against the cardiac isotype myosin (lower row, same secondary antibody).

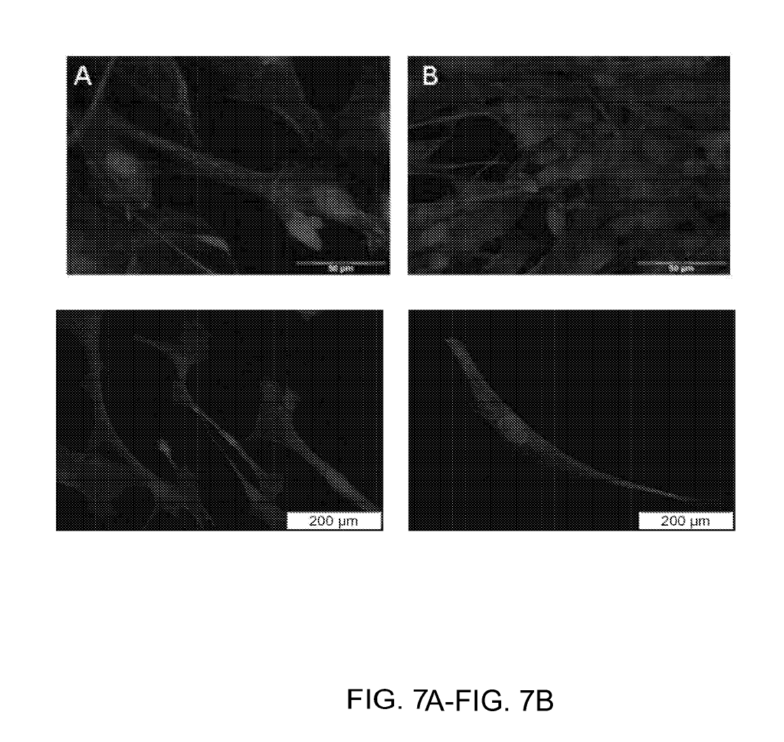

FIG. 8 shows fluorescent microscopy pictures of cells after myogenic induction with 5-azacytidine, which are positive for the antibodies against α-desmin (top, monoclonal mouse-anti-human desmin, Dako) and α-smooth muscle myosin (bottom, monoclonal mouse-anti-human myosin heavy chain (smooth muscle), Dako).

FIG. 9A-FIG. 9D shows an oil red 0 staining of human cells as indicator of adipogenic differentiation or lack thereof. FIG. 9A: Cardiac progenitor cells day 15; FIG. 9B: induced cardiac progenitor cells day 15; FIG. 9C: human MSC day 15; FIG. 9D: induced human MSC day 15.

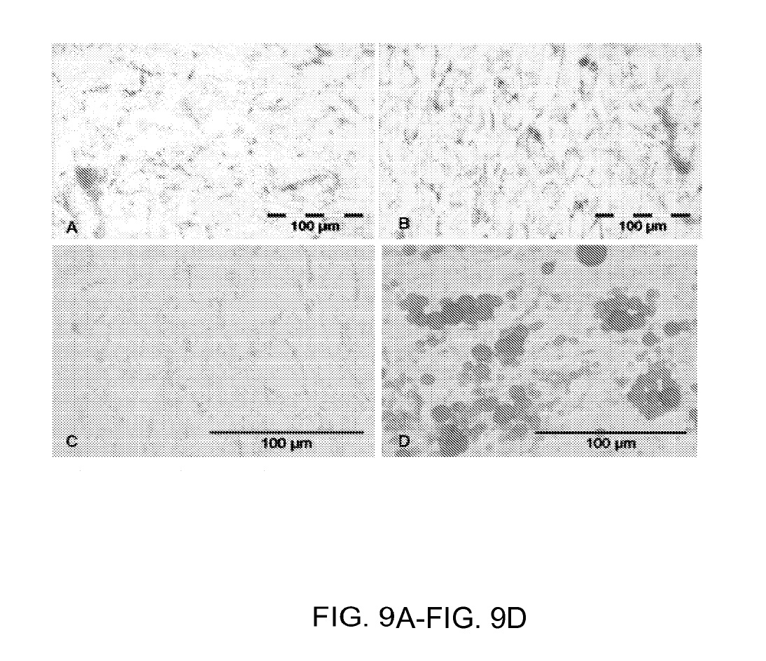

FIG. 10A-FIG. 10F exemplifies staining to confirm chondrogenesis or lack thereof in a patient at the end of induction (day 28); FIG. 10A shows the alcian blue staining, FIG. 10C the collagen type II, and FIG. 10E the collagen type I staining of the cell pellet without chondrogenic induction (check). FIG. 10B shows the alcian blue, FIG. 10D the collagen type II, and FIG. 10F the collagen type I staining cell pellet with chondrogenic induction.

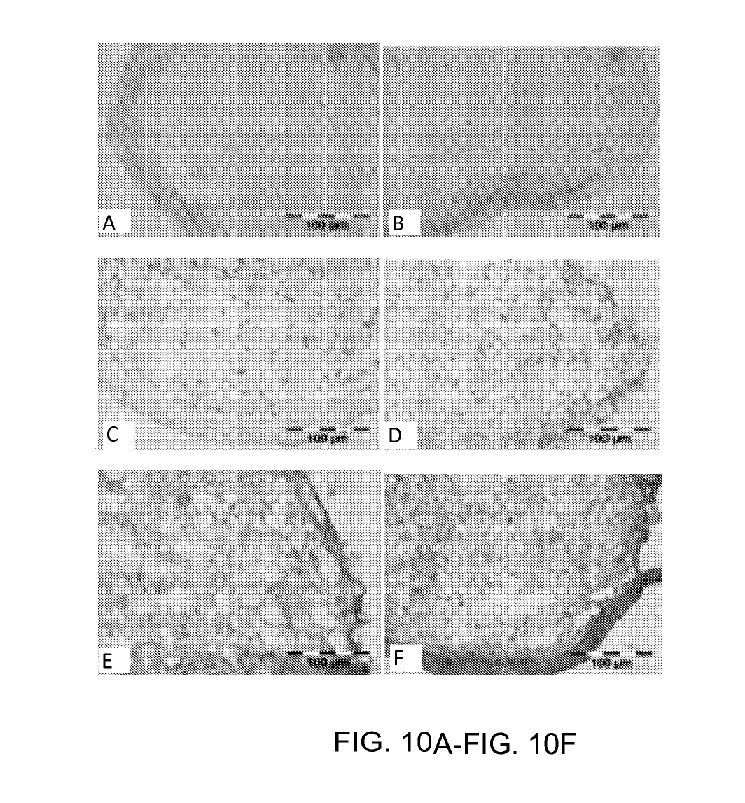

FIG. 11A-FIG. 11H shows histological confirmation of the formation or the absence of bone matrix by means of Kossa staining. FIG. 11A-FIG. 11C show non-induced control cultures of the patients 1-3 on day 28, FIG. 11D shows a non-induced human MSC control culture on day 28, FIG. 11E-FIG. 11G show osteogenically induced cultures of the patients 1-3 on day 28, FIG. 11H shows a clearly observable bone matrix in a osteogenically induced hMSC culture on day 28.

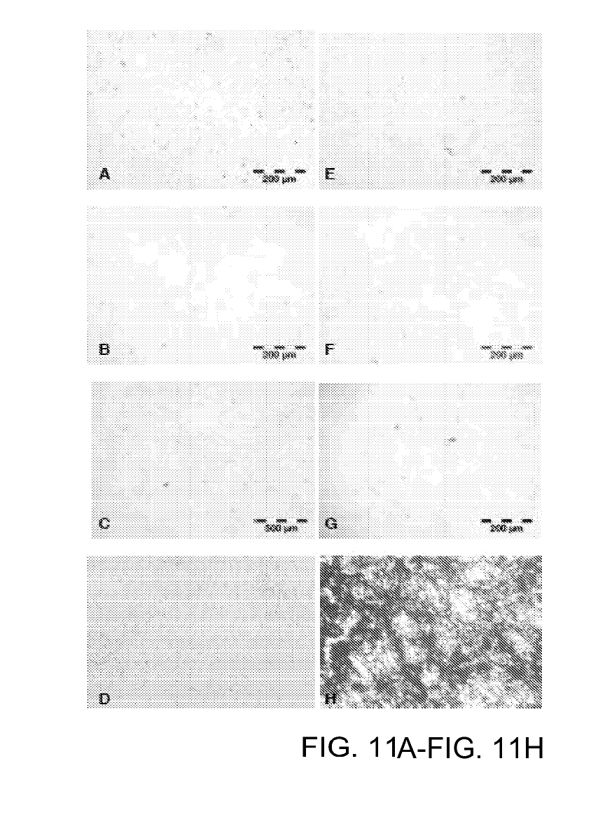

FIG. 12 shows the evaluation of an experiment for mixed lymphocyte reaction of 5 patients (n=5).

FIGS. 13A and 13B show the results of the FACS sorting. FIG. 13A, from the left to the right: unsorted, CD90 positive cells; CD90 negative cells.

In order to further illustrate the present invention and the advantages thereof, the following specific examples are given, it being understood that same are intended only as illustrative and in no way limiting.

### EXAMPLES

### Example 1

### Culture Conditions and Passage

Reprocessing of Biopsy Materials: □

Biopsy material obtained from a heart muscle was cut into pieces up to 5 mm³ in size, preferably 1-2 mm³ with a sterile scalpel and washed with PBS (phosphate-buffered isotonic solution of sodium chloride) (free of calcium and magnesium).

The tissue samples obtained in this way were digested 3×5 min at 37° C. with Trypsin/EDTA and 0.45 u/ml collagenase IV (Sigma Aldrich) in PBS (1:500 diluted, activity of the undiluted solution 0.125-0.15 u/ml; Biochrom AG, Berlin). After 5 minutes, the biopsy materials were transferred into a new trypsin-collagenase mixture, respectively. The supernatant was discarded and the pre-digested tissue was washed with IMDM (Iscove's Modified Dulbecco's Medium completed with 10% FBS (fetal bovine serum), 100 u/ml Penicillin, 100 μg/ml streptomycin, 2 mmol/L L-glutamine), afterwards the explants were cultivated in completed IMDM medium in a cell culture container having 9.6 cm² growth surface. Explants have to be fastened (“firmly pressed”) to the bottom of the culture container.

Depending on the explant (depending on the individual patient) fibroblast-like, adherent cells grow out after 7-15 days.

Harvest of Grown Cells: □

The explants/cells were carefully washed with PBS (explants should not □ detach), and then incubated for 3-5 minutes with 1 ml Trypsin/EDTA (0.05%/0.02%) per explant (culture panel size 9.6 cm², see above), and afterwards the digestion reaction was stopped with IDH medium (IMDM, DMEM, Ham F-12 Mix completed with 2% B27, 10 ng/ml epidermal growth factor, 20 ng/ml basic fibroblast growth factor, 3.3% FKS, 100 u/ml penicillin, 100 ug/ml streptomycin, 2 mmol/L L-glutamine).

Afterwards the medium with the detached cells was centrifuged for 5 minutes at 353 g, the supernatant discarded and the cells resuspended in IDH medium, and cultured in a total volume of 3 ml medium (IDH) in a 9.6 cm² culture container (conditions: 5% CO2, 37° C., 95% humidity).

Alternatively, a medium could be used containing 5% human serum instead of 3.3% FBS without B27. Alternatively, the commercially available medium Opti pro™ (Gibco, 12309) complemented with the corresponding amounts of serum, penicillin/streptomycin, 200 mM L-alanyl-L glutamine (Biochrom, K0302, 20 ml/L), EGF and FGF could be used.

The cells are passaged at a confluence of 70-90%. For this, the medium is removed, the cell layer is rinsed once with PBS, and the cells are trypsinized.

Then, 5,000-6,000 cells are sown per cm² cell culture container surface. The cells or cell preparations according to the invention were isolated up to four times from the same explant at intervals of 6-10 days (depending on the biopsy material).

### Example 2

### Staining

The trypsinized cells were washed with PBS/0.5% BSA. Afterwards □250,000 cells were incubated on ice for 15 minutes in 0.1 ml PBS/0.5% BSA and the corresponding antibody (AK). Fluorescein isothiocyanate (FITC) labeled, R-phycoerythrin (PE) labeled and allophycocyanin (APC) labeled mouse anti-human AK were used (see Table X). The cells were washed with PBS/0.5% BSA after staining. Apoptotic cells were labeled with propidiumiodide (PI, Sigma, Taufkirchen, Germany), in order to exclude them from evaluation. The analysis was conducted using the FACSCalibur device (Becton Dickinson, Heidelberg, Germany) and the evaluation performed with help of CellQuest Software (Becton Dickinson).

TABLE 1
Information concerning antibodies used

| Antibody | Dilution | Manufacturer | Order No. |
|---|---|---|---|
| FITC α human CD90 | 1:75 | Pharmingen | 555595 |
| FITC α human CD105 | 1:20 | Acris | SM1177F |
| APC α HumanCD117 | 1:20 | Invitrogen | CD11705 |
| PE α humanCD166 | 1:20 | Pharmingen | 559263 |
| FITC α humanCD45 | 1:100 | Pharmingen | 555482 |
| PE α humanCD34 | 1:50 | Pharmingen | 555822 |
| PE α humanCD73 | 1:20 | Pharmingen | 550257 |

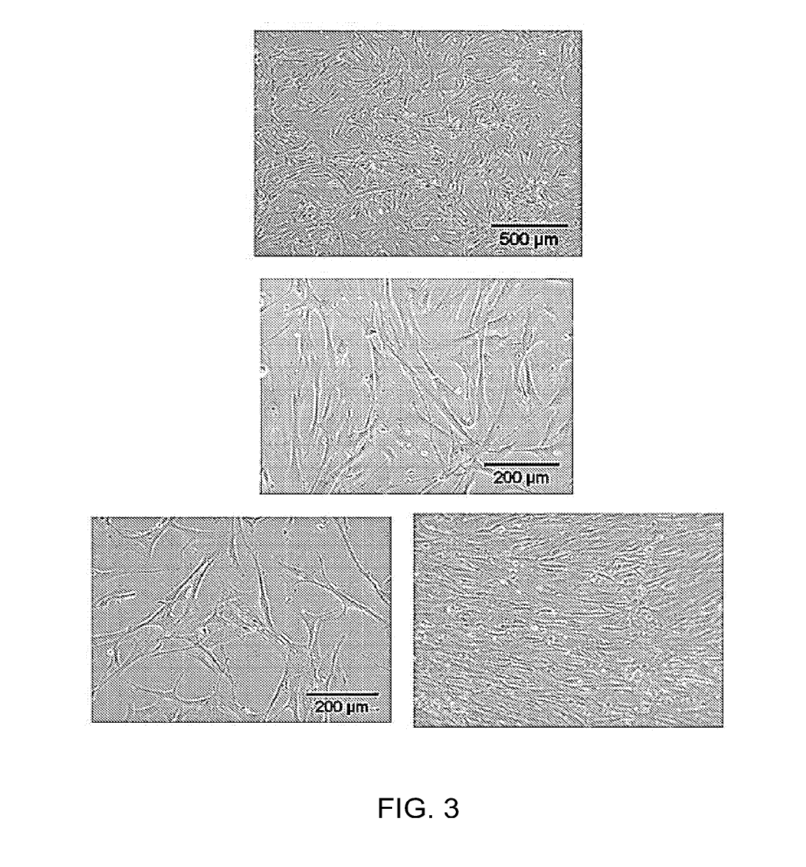

FIG. 2 shows in panels A and B adherent cells growing in culture from the biopsy material of (A: after 5 days in culture), which expand and adopt a fibroblast-like shape after some days (B: after 15 days in culture). C shows a cell culture of the harvested cells in passage 4 after 5 days in culture. D-I show in the FACS analysis, that cells are to the largest extent negative for CD90 (E and F) and CD117 (G) and positive for CD105 (H) and to the largest extent also positive for CD73 (I). The line shows the histogram of the negative (unstained) cell population, the histogram of the stained cells is depicted two-dimensionally.

The following Table 2 exemplifies parameter used in a measurement with the device “FACSCalibur” (Becton-Dickinson):

TABLE 2
Measurement parameters of the channels of the FACS device

| Param | Detector | Voltage | Amplification | Mode |
|---|---|---|---|---|
| P1 | forward scattering | E-1 | 3.27 | Lin |
| P2 | sidewards scattering | 424 | 1.00 | Lin |
| P3 | fluorescence channel1 | 505 | 1.00 | Log |
| P4 | Fl. 2 | 489 | 1.00 | Log |
| P5 | Fl. 3 | 590 | 1.00 | Log |
| P6 | Fl. 2-A | | 1.00 | Lin |
| P7 | Fl. 4 | 740 | | Log |

TABLE 3
Compensations of the channels of the FACS device were
Compensation

FL1 - 2.5% FL2
FL2 - 1.0% FL1
FL2 - 2.0% FL3
FL3 - 14.6% FL2
FL3 - 0.8% FL4
FL4 - 1.0% FL3

Primary threshold parameter: Fluorescence channel 1, value: 35 □

### Example 3

### Culture with 5-Azacytidine (Myogenic Induction, Myogenesis)

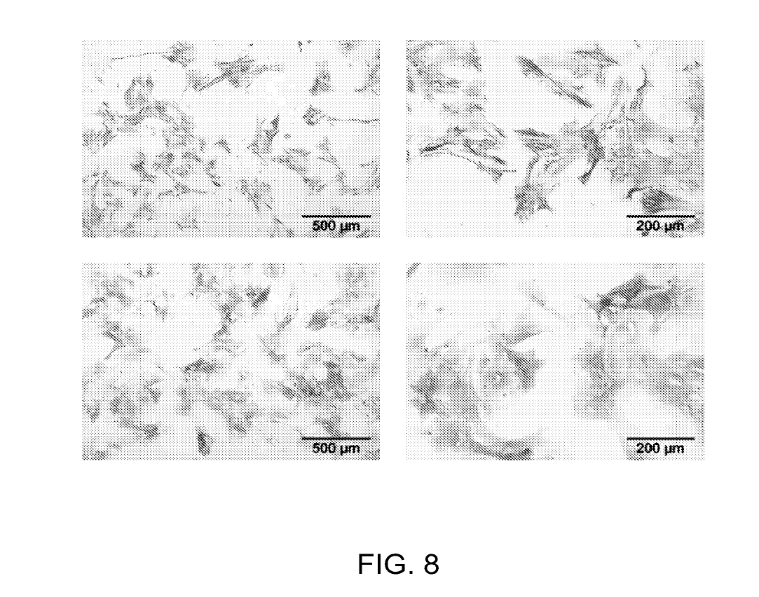

After stimulation with 5-azacytidine (24 h-20 μl/ml, 10 μM) and 4 week cultivation the cells were positive for α-desmin-antibodies and α-smooth muscle myosin-antibody (see FIG. 8).

(according to: Xu W, Zhang Z, Mesenchymal stem cells from adult human bone marrow differentiate into a cardiomyocyte phenotype in vitro, Exp Biol Med (Maywood). 2004 July; 229(7):623-31)

Differentiation in Fat/Bones/Gristles (“Multilineage”) □

When cells were induced in accordance with the modified protocols of □ Pittenger et al. (Pittenger et al., Multilineage potential of adult human mesenchymal stem cells. Science. 1999 Apr. 2; 284(5411):143-7) for the cells described here, they did not differentiate in to fat, bone and gristle. For this, the protocols of Pittenger et al. were adapted to the media used in example 1.

### Example 4

### Isolation of a Cell Population by Means of FACS Sorting

Cells were stained analogously to example 2, wherein: 10-15 million □ cells were labeled with αhumanCD90 FITC (BD) in a 1:100 dilution. The cells were washed with 10 ml PBS/BSA and prepared in a 1 ml PBS/BSA working volume.

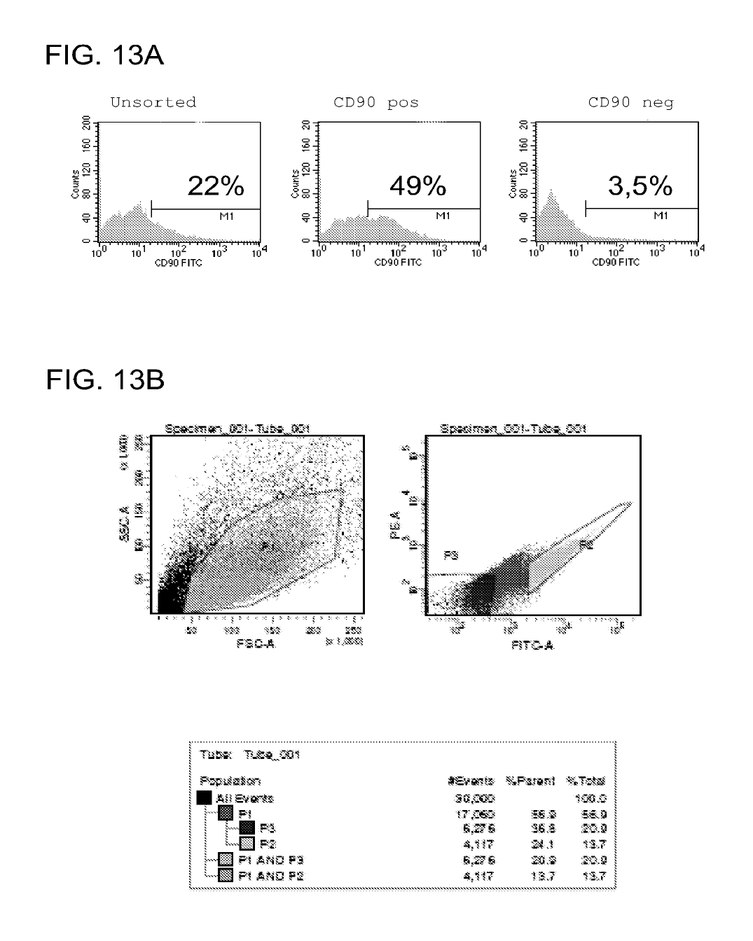

Cells stained in this way were sorted using known methods, for example by means of FACS sorting. The result of the sorting is shown in FIG. 13. The percentage values state the number of the CD90 positive cells. Before sorting 22% of the cells were CD90 positive, then, in the population sorted with respect to CD90, 49% of the cells were CD90 positive. The population sorted with respect to CD90 negative was 96.5% negative.

### Example 5

### Mixed Lymphocyte Reaction (MLR)

By means of the MLR, alloreactive T cells can be detected in the □ experiment. These are evidence that a rejection of alien tissue/alien cells will occur. In the MLR lymphocytes of an individual (donor A) are mixed with lymphocytes of another individual (donor B). When the T cells of the one individual recognize the MHC molecules of the other individual as alien, these T cells proliferate. The proliferation of donor A cells was prevented by means of treatment of the cells with mitomycin C (m), a cytostatic compound.

CAP cells exhibit immunomodulatory properties. As the MLR shows, they prevent proliferation of T cells at numbers as low as 5×10⁴ cells, and thus act as immunosuppressants. Thus, these cells behave similar to mesenchymal stem cells concerning the MLR. This is important evidence for the possibility of an allogeneic application of the cells.

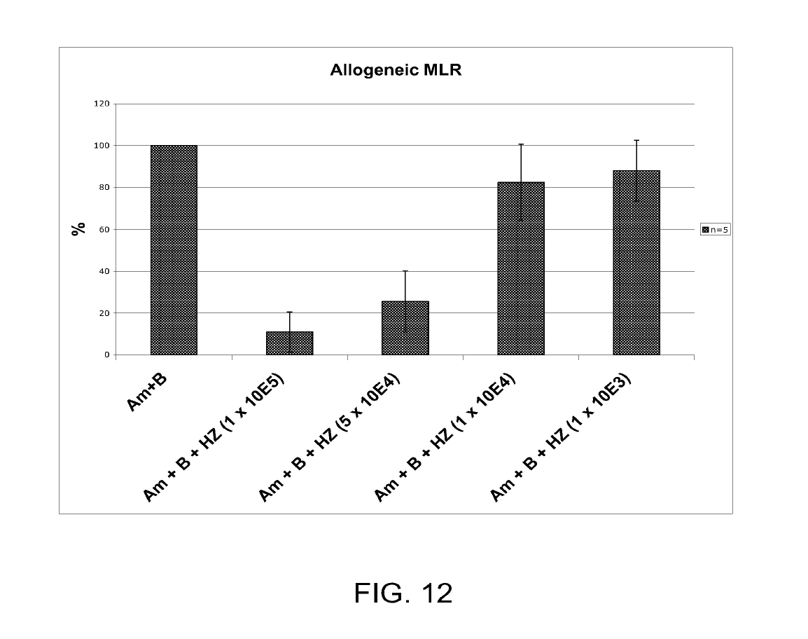

Different concentrations of heart cells (HZ) were mixed with mitomycin treated cells of donor A (Am). At a concentration of 5×10⁴ and 1×10⁵ heart cells, the proliferation and thus the immune reaction of the cells of donor B were suppressed. The immune reaction of the cells of donor A (mitomycin treated, Am) with the cells of donor B (see FIG. 12) served as a reference.

TABLE 4
Antibodies for the FACS analysis prior to MLR
BAB-9502

| AB denotation | Company | Product number |
|---|---|---|
| CD31 - PE | BD | 555446 |
| CD34 - PE | BD | 550761 |
| CD45 - FITC | BD | 555482 |
| CD73 - PE | BD | 550257 |
| CD80 - PE | BD | 557227 |
| CD86 - PE | BD | 555653 |
| CD90 - FITC | BD | 555595 |
| CD105 - PE | Caltag | MHCD10504-4 |
| HLA I - FITC | BD | 555552 |
| HLA II - FITC | BD | 555558 |
| CD 40 PE | Serotec | MCA1590PE |

While the invention has been described in terms of various preferred embodiments, those of skill in the pertinent art will appreciate that various modifications, substitutions, omissions, and changes may be made without departing from the spirit thereof. Accordingly, it is intended that the scope of the present invention be limited solely by the scope of the following claims, including equivalents thereof.

We claim:

1. A method for obtaining a cell preparation comprising isolated fibroblast-like cells that are CD90 negative; CD105 positive; and CD117 negative, the method comprising:
a) digesting a tissue sample obtained from mammalian heart muscle tissue in a time limited digestion by one or more connective tissue digesting enzymes;
b) culturing the time limited digested tissue under conditions suitable for culture of mammalian cells in cell culture medium, in a cell culture container having a solid surface;
c) detaching cells grown out of the tissue sample adhering to the solid surface in a passage step by means of limited proteolysis, isolating the detached cells and culturing the detached cells;
e) wherein the cell culture medium of either the first cell culture step, the passage step or of both steps does not contain cardiotrophin, thrombin or mercaptoethanol, thereby obtaining the cell preparation.

2. The method of claim 1, wherein step (c) is repeated at least twice.

3. The method of claim 1, further comprising stimulating the cells with 5-azacytidine before, during or after the step (b).

4. The method of claim 1, wherein the connective tissue digesting enzyme comprises at least one of trypsin-EDTA or collagenase IV, and wherein the time limited digestion is less than 10 minutes at an activity of 0.05 to 0.25 u/500 ml for trypsin and/or 0.2 to 4.5 u/ml for collagenase IV.

5. The method of claim 1, wherein step (b) has a duration of 7 to 15 days.

6. The method of claim 1, wherein step (c) is conducted once the cells adhering to the solid surface are at least 70% confluence.

7. The method of claim 1, further comprising subjecting the cell preparation to a purification step, comprising:
contacting the cells in the cell preparation with molecules which are capable of binding to specific cell surface markers; and
separating those cells to which molecules capable of binding to specific cell surface markers have bound.

8. The method of claim 7, wherein molecules capable of binding to specific cell surface markers are one or more antibodies against CD90, CD105, CD166, CD34, or CD45.

9. The method of claim 7, wherein the step of separating the bound cells comprises:
contacting the cells in the cell preparation with molecules capable of binding to the molecules bound to the specific cell surface markers, wherein the contacting molecules are linked to magnetic particles;
applying a magnetic field to separate the bound cells.

10. The method of claim 7, wherein the step of separating the bound cells comprises fluorescence activated cell sorting.
